(19) United States
(12) Patent Application Publication
(10) Pub. No.: US 2024/0197686 A1
(43) Pub. Date: Jun. 20, 2024

(54) ANTI-TUMORAL EFFECTIVE BENZIMIDAZOLE ZINC COMPLEXES

(71) Applicant: INONU UNIVERSITESI REKTORLUGU, Malatya (TR)

(72) Inventors: Elif APOHAN, Malatya (TR); Ozfer YESILADA, Malatya (TR); Hasan KUCUKBAY, Malatya (TR); Ozgur KATRANCIOGLU, Malatya (TR); Hakan PARLAKPINAR, Malatya (TR); Mehmet GUL, Malatya (TR); Ulku YILMAZ, Malatya (TR); Yusuf BARAN, Izmir (TR)

(21) Appl. No.: 18/287,185

(22) PCT Filed: Nov. 10, 2022
(86) PCT No.: PCT/TR2022/051279

§ 371 Date: Oct. 17, 2023

(51) Int. Cl. A61K 31/4184; A61P 35/00

(52) U.S. Cl. CPC A61K 31/4184; A61P 35/00

(57) ABSTRACT

Invention relates to use of antitumoral effective benzimidazole metal complex containing zinc having antitumoral effect, in treatment of cancer. The invention particularly relates to use of 1-(4-chlorobenzyl)benzimidazole and 1-(4-methylbenzyl)benzimidazole zinc complexes in in vivo cancer treatment

### TECHNICAL FIELD

The invention relates to use of anti-tumour effective benzimidazole metal complexes containing zinc having antitumoral effect in treatment of cancer.

The invention particularly relates to use of 1-(4-chlorobenzyl) benzimidazole and 1-(4-methylbenzyl) benzimidazole zinc complexes in in vivo cancer treatment

### BACKGROUND OF THE INVENTION

Lung cancer is in the second place after breast cancer in women and prostate cancer in men in terms frequency of occurrence. This type of cancer corresponds to 14% in men and 13% in women with new cancer diagnosis in 2018 in the USA.

According to cancer statistics in Turkey published by Turkish Ministry of Health lung, cancer is in the first place based on 2016 data.

Today it is seen that the cancer medicines used for treatment purposes are generally platin-containing organometallic or inorganic platin compounds.

Ototoxicity is a cellular degeneration and function disorder developed in cochlea and vestibular organ by various medicines and chemicals. Cisplatin [(cis-diamminedichloroplatinum II)] is an antineoplastic agent particularly used in treatment of several malign diseases such as head-neck tumours, urogenital system, central nervous system, respiratory system and esophageal cancer in adults and children. Cisplatin's other essential dose restrictive side effects other than nephrotoxicity and irreversible ototoxicity are neurotoxicity, gastrointestinal system and bone marrow toxicity. Cisplatin shows its ototoxic effect by reactive oxygen products and DNA damage.

Cisplatin's disadvantage is its display of neuro and nephrotoxic feature. In the paper by Prof. Dr. Ismail Savaq, professor at Department of Chest Diseases and Tuberculosis at Ankara University School of Medicine entitled Treatment of Lung Cancer, studies on single agent Cisplatin and new agents in combination with cisplatin show that the combination gives better results. While surviving achieved by cisplatin alone is 6 months, surviving after treatment with combined vinorelbine and cisplatin prolongs to 8 months. The studies made with cisplatin, combination of cisplatin and gemsitabin report that surviving is 7 weeks longer.

As also seen in the studies of related and study conducted under this invention, in case where cisplatin is used alone as positive control, the surviving period is short and it necessitates need for new effective agents. Also as stated in paper by Shaloam Dasari and Paul Bernard Tchounwou’ entitled “Cancer therapy: Molecular mechanisms of action” (European Journal of Pharmacology, 740(2014)364-378) while cisplatin shows strong cytotoxic antitumor activity in clinical experiments, it has additional side effects such as renal toxicity, nausea, vomiting.

The paper by Sara A. Aldossary published in 2019 in Biomedical & Pharmacology Journal, March. Vol. 12(1), p. 7-15 and entitled “Review on Pharmacology of Cisplatin: Clinical Use, Toxicity and Mechanism of Resistance of Cisplatin” reports that cisplatin is a chemotherapeutic agent used in treatment of several cancers such as lung cancer, head and neck cancers. In addition, it is emphasized that cisplatin displays resistance at certain level including enhancement in repair of damaged DNA and reduction in cytosolic activation and intracellular collection of medicine causes side effects such as neurotoxicity, nephrotoxicity, cardiotoxicity and hepatoxicity.

In addition to the stated side effects of cisplatin, as also indicated under the study disclosed under the invention, it was discovered that it causes histopathological damages in lung, liver and kidney tissues.

In the literature Turkish patent numbered TR2015/10411 based on previous studies by inventors made inventions in the related topic is seen. The patent is on coordination complexes having anticarcinogen and antimicrobial effectiveness. According to invention 1-substitued benzimidazole zinc and cobalt complexes have high biological effectiveness. The patent is granted for 3 compounds based on the results of in vitro studies. However, the present patent is based on the data obtained from in vivo studies. 2 compounds are characterized to decrease the tumour size developed by lung cancer cells by intraperitoneal injection administered to rats. Cisplatin used as the control group caused tissue damages while the stated two compounds did not cause tissue damage.

As a result, due to above-described disadvantages and inadequacy of existing solutions it has been necessary to make development in the related art.

### BRIEF DESCRIPTION OF THE INVENTION

The present invention relates to anti-tumour effective benzimidazole zinc complexes meeting the needs mentioned above, eliminating all disadvantages and providing some additional advantages.

Primary purpose of the invention is to provide decrease in tumour size subject to application of benzimidazole metal complex compounds containing zinc.

Another purpose of the invention is to develop a cancer treatment not causing histopathological damage in lung, liver and kidney tissues and not developing toxic effect.

In order to achieve above mentioned purposes, benzimidazole metal complex compounds containing zinc, providing anti-tumour effect are used in cancer treatment.

The structural and characteristics features of the invention and all advantages will be understood better in detailed descriptions with the figures given below and with reference to the figures, and therefore, the assessment should be made taking into account these figures and detailed explanations.

### BRIEF DESCRIPTION OF THE DRAWINGS

FIG. 1 is the view of experimental analysis made with control group.
a. Normal lung histology, bronchiole (b). H-E, ×10
b. Control group: Normal lung histology, alveoli (a), bronchiole (b). H-E, ×20
c. Control group: Normal lung histology, alveoli (a), bronchiole (b). H-E, ×40
d. Control group: Normal kidney histology, glomerulus (arrow). H-E, ×10
e. Control group: Normal kidney histology, glomerulus (arrow). H-E, ×20
f. Control group: Normal kidney histology, glomerulus (G), distal tubule (d) proximal tubule (p). H-E, ×40
g. Control group: Normal liver histology. H-E, ×10
h. Control group: Normal liver histology. Central ven (S). Portal triad (arrow caps) H-E, ×20
i. Control group: Normal liver histology, hepatoxicity (arrow), central ven (S). H-E, ×40

FIG. 2 is the view of experimental analysis made with DMSO group.
a: Normal lung histology, bronchiole (b). H-E, ×10
b: Normal lung histology, alveolol (a), bronchiole (b). H-E, ×20
c: Normal lung histology, alveolol (a). H-E, ×40
d: Normal kidney histology, glomerulus (arrow). H-E, ×10
e: Normal kidney histology, glomerulus (arrow). H-E, ×20
f: Normal kidney histology, glomerulus (G). Distal tubule (d), proximal tubule (p). H-E, ×40
g: Normal liver histology. H-E, ×10
h: Normal liver histology. Central ven (S). H-E, ×20
i: Normal liver histology, hepatocyte (arrow), central ven (S). H-E, ×40

FIG. 3 is the view of experimental analysis made with cisplatin group.
a: Lung histology, bronchiole (b) in normal histological view. Secretion (aster) in bronchiole lumen, vascular congestion (arrow). H-E, ×10
b: Normal lung histology, bronchiole (b), alveoli (a) H-E, ×20
c: Normal lung histology, alveol (a). H-E, ×40
d: Kidney, collapse in glomerular (arrow), vascular congestion (arrow). H-E, ×10
e: Kidney, narrowing in glomerular Bowman's space (arrow), vascular congestion (aster). H-E, ×20
f: Kidney, narrowing in glomerular Bowman's space (arrow), tubule epithelial damage (aster). H-E, ×40
g: Liver, hemorage (aster) in parenchymal area, vascular congestion (arrow), eosinophilic concentration in hepatocyte cytoplasm and heterochromia increase in nucleus (+). H-E, ×10
h: Liver, dark eosinophilic cytoplasm and heterochromatic pycnotic nuclei hepatocytes (arrow) H-E, ×20
i: Liver, vacuolisation (aster) in hepatocytes (aster). H-E, ×40

FIG. 4 is the view of experimental analysis made with 1. Compound group.
a: Normal lung histology, bronchiole (b). H-E, ×10
b: Lung, alveoli (a). b bronchiole (b), thickening in alveoli wall (arrow) H-E, ×20
c: Normal lung histology, alveolol (a), bronchiole (b). H-E, ×40
d: Normal kidney histology, glomerulus (arrow). H-E, ×10
e: Normal kidney histology, glomerulus (arrow). H-E, ×20
f: Normal kidney histology, glomerulus (G), distal tubule (d) proximal tubule (p). H-E, ×40
g: Normal liver histology. H-E, ×10
h: Normal liver histology. Central ven (S). H-E, ×20
i: Normal liver histology, hepatocyte (arrow), central ven (S). H-E, ×40

FIG. 5 is the view of experimental analysis made with 5. Compound group.
a: Normal lung histology, bronchiole (b). H-E, ×10
b: Lung, alveoli (a). b bronchiole (b), thickening in alveoli wall (arrow). H-E, ×20
c: Lung, alveoli (a). b bronchiole (b), thickening in alveoli wall (arrow). H-E, ×40
d: Kidney, glomerular collapse (arrow). Vascular congestion (aster), capsule bordered in kidney tissue adjacent (arrow heads) tumour tissue (+) H-E, ×10
e: Kidney, dilation in glomerular Bowman's space (arrow head), tubule epithelia damage and tubule lumen degenerated cells (arrow). H-E, ×20
f: Kidney, dilation and accumulation in glomerulus Bowman's space (arrow), tubule epithelia damage (aster) H-E, ×40
g: Liver, eosinophilic concentration in hepatocyte cytoplasm and heterochromia increase (aster) in nucleus. H-E, ×10
h: Liver, vascular congestion (aster), inflammatory cell infiltration periportal area (+), hepatocyte cytoplasm eosinophilic concentration and heterochromatic pycnotic nuclei (arrow). H-E, ×20
i: Liver, cytoplasmic vacuolisation (aster) in hepatocytes. H-E, ×40

FIG. 6 is a graphic indicating the albumin level of nude rats administered with benzimidazole metal complex compounds containing zinc (Compounds 1 and 5)

FIG. 7 is a graphic indicating the ALT level of nude rats administered with benzimidazole metal complex compounds containing zinc (Compounds 1 and 5)

FIG. 8 is a graphic indicating the AST level of nude rats administered with benzimidazole metal complex compounds containing zinc (Compounds 1 and 5)

FIG. 9 is a graphic indicating the BUN level of nude rats administered with benzimidazole metal complex compounds containing zinc (Compounds 1 and 5)

FIG. 10 is the graphic indicating the creatinine level of nude rats administered with benzimidazole metal complex compounds containing zinc (1 and 5 compound)

FIG. 11 shows the effect of DMSO on p53, Caspase 3, Bak, Bax and Aktin protein expressions in Lung (A), Kidney (B), Liver (K), Tumour (T) and Peritone Omentum (P/O) tissues

FIG. 12 shows the effect of compound 1 on p53, Caspase 3, and Aktin protein expressions in Lung (A), Kidney (B), Liver (K), Tumour (T) and Peritone Omentum (P/O) tissues

FIG. 13 shows the effect of compound 5 on p53, Caspase 3, Bak and Aktin protein expressions in Lung (A), Kidney (B), Liver (K), Tumour (T) and Peritone Omentum (P/O) tissues

The drawings are not necessarily to be scaled and the details not necessary for understanding the present invention might have been neglected. In addition, the components which are equivalent to great extent at least or have equivalent functions at least have been assigned the same number.

### DETAILED DESCRIPTION OF THE INVENTION

In this detailed description, the subject of antitumour effect benzimidazole zinc complex is described in a manner not forming any restrictive effect and only for purpose of better understanding of the topic.

The invention is the use of antitumour effective benzimidazole metal complex containing zinc having antitumour effect, in treatment of cancer. Compounds of the invention are selected from 1-(4-chlorobenzyl) benzimidazole and 1-(4-methylbenzyl) benzimidazole zinc complexes.

The invention particularly has potential of use in in vivo cancer treatment.

Pharmaceutical compositions comprising zinc based benzimidazole metal complex compounds of the subject of invention, are used in treatment of cancer

Experimental studies conducted regarding the invention and results therefrom are explained below:

Effect of Benzimidazole Metal Complex Compounds Containing Zinc (1^{st} and 5^{th} Compound) on Size and Weight of Tumour in Tumour Developed Nude Rats

A549 cells reproduced in cell culture as 1×10⁷ cells were injected to abdominal cavity of nude rats of which weights were measured. After one week DMSO, 1^{st} and 5^{th} compound application was made and weights and tumour sizes were measured once a week. Tumour measurements were measured by manual compass as Length (head-tail)-Width (left-right) and Height (abdominal wall-outside of skin) and recorded in this sequence (Length×Width×Height). The following Table 1-A and Table 1-B show weight and tumour sizes measured after DMSO application.

TABLE-1A
Weights (g) and tumour sizes (mm) of DMSO applied
nude rats after A549 cells injection

| | DMSO | | | | | | | |
|---|---|---|---|---|---|---|---|---|
| | EMPTY | | LEFT INCISION | | RIGHT INCISION | | LEFT CUT | |
| | | Tumour | | Tumour | | Tumour | | Tumour |
| Week | Gram | Size | Gram | Size | Gram | Size | Gram | Size |
| Cell | 27.3 | 0 × 0 × 0 | 27.8 | 0 × 0 × 0 | 23.5 | 0 × 0 × 0 | 29.4 | 0 × 0 × 0 |
| Injection | | | | | | | | |
| 1. | 28.6 | 4 × 5 × 2 | 27.6 | 5 × 2 × 1 | 29.2 | 3 × 3 × 2 | 30.5 | 10 × 1 × 1 |
| 2. | 31.3 | 2 × 2 × 1 | 31.2 | 2 × 1 × 1 | 29.5 | 4 × 6 × 3 | 28.4 | 1 × 1 × 1 |
| | | 3 × 4 × 1 | | (3 times) | | | | 1 × 1 × 1 |
| | | | | | | | | 8 × 3 × 2 |
| 3. | 31.1 | 4 × 5 × 3 | 32.1 | 3 × 2 × 1 | 32.4 | 4 × 2 × 1 | 31.7 | 10 × 2 × 1 |
| 4. | 33.3 | 4 × 1 × 1 | 33.3 | 3 × 2 × 1 | 34.1 | 2 × 2 × 1 | 31.2 | 4 × 7 × 3 |
| | | | | 2 × 2 × 1 | | | | |

TABLE-1B
Weights (g) and tumour sizes (mm) of DMSO applied nude rats after A549 cells injection

| | DMSO | | | | | | | | | |
|---|---|---|---|---|---|---|---|---|---|---|
| | | | | | | | Right Left | | | |
| | Empty | | Right incision | | Left incision | | Incision | | Right cut | |
| | | Tumour | | Tumour | | Tumour | | Tumour | | Tumour |
| Week | Gram | Size | Gram | Size | Gram | Size | Gram | Size | Gram | Size |
| 1. | 32.9 | 3 × 2 × 1 | 28.4 | 2 × 3 × 2 | 28.3 | 4.5 × 2 × 2 | EX | | 26.8 | 5 × 2 × 1 |
| 2. | 33.4 | 3 × 3 × 2.5 | 19.6 | 1 × 1 × 1 | 23.7 | 3.5 × 3 × 2 | | | 20.4 | 5 × 2 × 1.5 |
| 3. | 34.4 | 4 × 4 × 4 | 26.4 | 3 × 2 × 1 | 29.1 | 3.5 × 2 × 2 | | | 26.5 | 6.5 × 3 × 2 |
| 4. | 34.8 | 5 × 5 × 5 | 29 | 7 × 4 × 3 | 30.9 | 5.5 × 5 × 4 | | | 31.9 | 4 × 3 × 2 |
| 5. | 33.2 | 6 × 7 × 5 | 32.1 | 3 × 2 × 1 | 30.4 | 5 × 4 × 2.5 | | | 29.4 | 4 × 4 × 3 |

Table 2-A and Table-2B show results of study where 1^{st} compound was administered. During the week following administration of compound it was observed that tumour sizes reduced and in following weeks increase in tumour size was observed.

TABLE 2-A
Weights (g) and tumour sizes (mm) of nude rats administered 1^{st} compound after A549 cells injection

| | Compound no. 1 | | | | | | | | | | | | | |
|---|---|---|---|---|---|---|---|---|---|---|---|---|---|---|
| | | | | | | | | | | | RIGHT LEFT | | RIGHT LEFT | |
| | EMPTY | | LEFT INCISION | | RIGHT INCISION | | LEFT CUT | | RIGHT CUT | | CUT | | CUT | |
| | | Tumour | | Tumour | | Tumour | | Tumour | | Tumour | | Tumour | | Tumour |
| Week | Gram | Size | Gram | Size | Gram | Size | Gram | Size | Gram | Size | Gram | Size | Gram | Size |
| Cell | 27.5 | 0 × 0 × 0 | 24 | 0 × 0 × 0 | 27 | 0 × 0 × 0 | 27 | 0 × 0 × 0 | 27.6 | 0 × 0 × 0 | 25 | 0 × 0 × 0 | 26.1 | 0 × 0 × 0 |
| Injection | | | | | | | | | | | | | | |
| 1. | 28.9 | 6 × 4 × 3 | 25.2 | 4 × 3 × 2 | 28.4 | 7 × 5 × 3 | 28.4 | 4 × 4 × 2 | 28.9 | 5 × 3 × 2 | 26.6 | 4 × 3 × 2 | 27.5 | 5 × 3 × 1 |
| 2. | 29.3 | 1 × 1 × 1 | 29.2 | 4 × 2 × 1 | 26.4 | 5 × 3 × 1.5 | EX | | | | 27.6 | 3 × 3 × 3 | 29 | 4 × 2 × 1 |
| 3. | 27.6 | 3 × 2 × 1 | 31.6 | 5 × 3 × 2 | 28.2 | 4 × 4 × 2 | | | | | 28.2 | 4 × 4 × 2 | 30.3 | 6 × 3 × 2 |
| | | | | | | 2 × 2 × 1 | | | | | | | | |
| 4. | 29.6 | 4 × 3 × 1 | 30.7 | 7 × 5 × 1 | 28.4 | 9 × 5 × 1 | | | | | 28.7 | 5 × 5 × 1 | 29.7 | 8 × 5 × 1 |

TABLE 2 B
Weights (g) and tumour sizes (mm) of nude rats administered 1^{st} compound after A549 cells injection

| | 1. Compound | | | | | | | | | |
|---|---|---|---|---|---|---|---|---|---|---|
| | | | | | | | Right Left | | | |
| | Empty | | Right incision | | Left incision | | Incision | | Right cut | |
| | | Tumour | | Tumour | | Tumour | | Tumour | | Tumour |
| Week | Gram | Size | Gram | Size | Gram | Size | Gram | Size | Gram | Size |
| 1. | 32.6 | 7 × 3 × 3 | 36.5 | 5 × 4 × 3 | 37.7 | 5 × 2 × 1.5 | 32.4 | 4 × 3 × 2 | 36 | 3.5 × 4 × 3 |
| 2. | 33.4 | 6 × 3.5 × 2 | 36.4 | 3 × 2.5 × 1 | 39.1 | 2.5 × 2 × 1 | 33.9 | 3.5 × 2 × 2 | 37 | 3 × 2 × 2 |
| 3. | 32.5 | 7 × 6 × 4 | 36 | 5 × 5 × 4 | 37.3 | 3 × 2 × 1 | 33.2 | 7 × 4 × 3 | 36.2 | 4 × 4 × 2 |
| 4. | 31.9 | 8 × 7 × 4 | 36.6 | 8 × 4 × 4 | 37 | 3 × 2 × 1 | 33.4 | 5 × 4 × 4 | 36.8 | 3 × 3 × 2 |
| 5. | 32.2 | 7 × 7 × 4 | 35.1 | 7 × 5 × 3 | 36.7 | 1 × 1 × 1 | 32.3 | 7 × 4 × 3.5 | 33.5 | 2 × 2 × 2 |

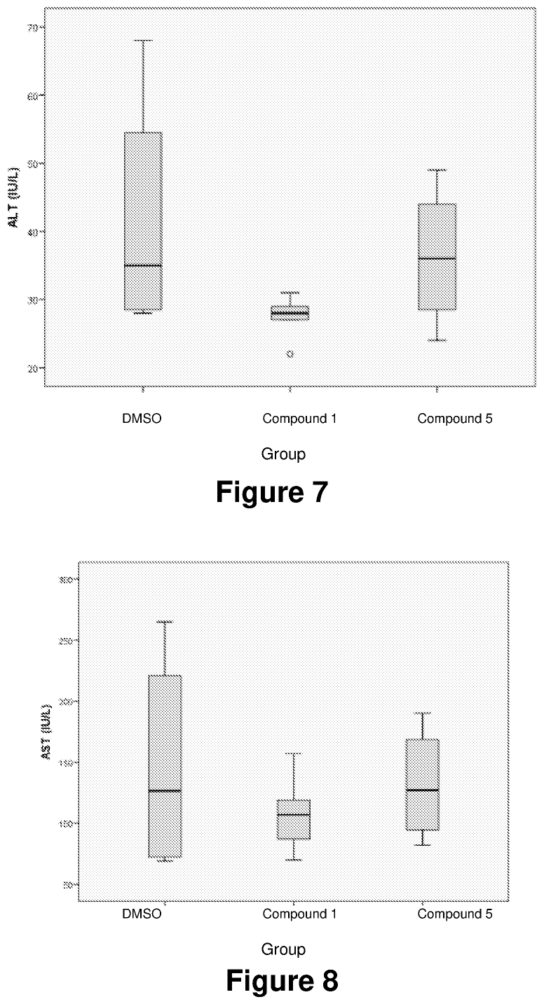

Table 3-A and 3-B show results of study conducted on effect of 5^{th} compound on tumour size in nude rats. Tumour size decreases in the week following application of the compound (FIG. 7).

TABLE 3-A
Weights (g) and tumour sizes (mm) of nude rats administered 5th compound after A549 cells injection

| | Compound no. 5 | | | | | | | | | | | |
|---|---|---|---|---|---|---|---|---|---|---|---|---|
| | | | | | RIGHT | | | | | | RIGHT LEFT | |
| | EMPTY | | LEFT INCISION | | INCISION | | LEFT CUT | | RIGHT CUT | | CUT | |
| | | Tumour | | Tumour | | Tumour | | Tumour | | Tumour | | Tumour |
| Week | Gram | Size | Gram | Size | Gram | Size | Gram | Size | Gram | Size | Gram | Size |
| Cell | 27.3 | 0 × 0 × 0 | 27.8 | 0 × 0 × 0 | 23.5 | 0 × 0 × 0 | 29.4 | 0 × 0 × 0 | 27.8 | 0 × 0 × 0 | 27.5 | 0 × 0 × 0 |
| energy | | | | | | | | | | | | |
| 1. | 27.6 | 6 × 3 × 2 | 28.3 | 5 × 3 × 2 | 23.9 | 2 × 1 × 1 | 29.8 | 3 × 2 × 1 | 28.3 | 4 × 3 × 2 | 27.8 | 3 × 3 × 1 |
| | | | | | | | | 4 × 3 × 1 | | | | |
| 2. | 28.6 | 4 × 3 × 1 | 28.1 | 8 × 2 | 26.3 | 1 × 1 × 1 | 30.5 | 3 × 4 × 1 | 28.2 | 5 × 6 × 1 | 26.9 | 6 × 3 × 2 |
| | | | | (with | | 1 × 1 × 1 | | 3 × 2 × 1 | | | | |
| | | | | nodule) | | 1 × 1 × 1 | | 2 × 1 × 1 | | | | |
| | | | | 6 × 2 | | | | 1 × 1 × 1 | | | | |
| 3. | 28.9 | 2 × 2 × 1 | 28.2 | 3 × 2 × 1 | 29.1 | 1 × 1 × 1 | 31.4 | 3 × 2 × 1 | 28.5 | 3 × 3 × 1 | 28.8 | 2 × 1 × 1 |
| | | | | 4 × 2 × 1 | | | | 2 × 2 × 1 | | 3 × 2 × 1 | | |
| | | | | | | | | 1 × 1 × 1 | | | | |
| 4. | 29.3 | 3 × 3 × 2 | 29.9 | 5 × 2 × 2 | 28.8 | 1 × 1 × 1 | 32.9 | 3 × 1.5 × 1 | 29.2 | 3 × 3 × 2 | 29.8 | 3 × 2 × 2 |
| | | | | 3 × 2 × 3 | | | | 1.5 × 1.5 × 1 | | 3 × 3 × 2 | | |
| | | | | | | | | 1 × 1 × 1 | | | | |

TABLE 3 B
Weights (g) and tumour sizes (mm) of nude rats administered 5th compound after A549 cells injection

| | 5. Compound | | | | | | | | | |
|---|---|---|---|---|---|---|---|---|---|---|
| | Empty | | Right incision | | Left incision | | Right Left Incision | | Right cut | |
| | | Tumour | | Tumour | | Tumour | | Tumour | | Tumour |
| Week | Gram | Size | Gram | Size | Gram | Size | Gram | Size | Gram | Size |
| 1. | 32 | 5 × 3 × 1.5 | 32.4 | 4 × 3 × 3 | 32 | 3 × 3 × 1.5 | 29.3 | 4 × 5 × 3 | 25.9 | 3.5 × 3 × 2.5 |
| 2. | 31.2 | 5.5 × 3 × 2 | 26.3 | 2.5 × 3 × 2 | 30.9 | 3 × 2.5 × 2 | 28.6 | 5 × 6 × 4 | 31.4 | 4 × 2.5 × 2 |

| | | | | | | | | | |
|---|---|---|---|---|---|---|---|---|---|
| 3. | 31.1 | 6 × 3 × 2 | 31.8 | 6 × 3.5 × 3.5 | 34 | 4 × 3.5 × 3 | 29.6 | 7.5 × 7 × 4 | EX |
| 4. | 31.5 | 4 × 3 × 3 | 33.6 | 7 × 3.5 × 3 | 33.7 | 4 × 5 × 3 | 31.8 | 9 × 7 × 7 | EX |
| 5. | 28.7 | 0 | 29.6 | 0 | 32 | 3.5 × 2 × 2 | 31.6 | 10 × 9 × 8.5 | EX |

### Effect of Benzimidazole Metal Complex Compounds Containing Zinc (Compounds 1 and 5) on Tissues of Tumour Developed Nude Rats

After saccharification operation; lung, liver, kidney tissues were taken and 10% were fixed in formaldehyde, and the sections prepared after their histological tissue follow-up procedures were examined with a light microscope.

### Control Group:

Lung, liver and kidney tissue cuts of control group were concluded as normal histological structure and view (FIG. 1).

### Group DMSO:

Similar to control group; lung, liver and kidney tissue sections of DMSO group were concluded as normal histological structure and view (FIG. 2).

### Cisplatin Group:

Secretion accumulations and vascular congestion were detected in bronchiole lung sections of cisplatin group and also epithelial damage of minimal level was observed. Other than these findings, lung parenchyma was assessed in normal histological structure.

Glomerular collapse and vascular congestion was noticed commonly in kidney cuts of cisplatin group. Some glomerulus was also discovered to have clearly narrowed Bowman's space. Epithelia cell damage of various levels was observed in kidney tubules. Liver sections of cisplatin group were observed to have hemorrhagic fields and vascular congestion in parenchyma. Some parenchyma fields were noticed to have clear cytoplasmic eosinophil increase in hepatocytes and heterochromatic pycnotic nuclei structure. In addition, intracytoplasmic vacuolisation was observed in hetatocytes in some places (FIG. 3).

### 1. Compound Group:

Although lung tissue cuts were observed in normal histological structure in general, some fields were noticed to have thickened alveoli walls.

The examined kidney sections were noticed to have damage views of minimal level in some proximal and distal tubule epithelia cells. Particularly apical cytoplasm fields. Collapse of minimal level in glomerular and narrowing in Bowman's space were observed. Inflammatory cell infiltration of light level was discovered in focal fields in kidney parenchyma.

Although liver parenchyma was observed in normal histological structure in examined liver sections, in some hepatocytes cytoplasm eosinophile increase in minimal level was noticed. Also hydropic views of minimal level were observed in some hepatocytes (FIG. 4).

### 5. Composite Group:

Lung tissue sections of 5^{th} composite group were observed to be in normal histological structure.

Kidney tubule and glomerular structures were observed to be in normal histological structure in the examined kidney sections but collapses of minimal level were observed in some glomeruli.

5. Liver sections of compound group were considered in normal histological structure (FIG. 5).
Effect of Benzimidazole Metal Complex Compounds Containing Zinc (1^{st} and 5^{th} Compound) on Blood Parameters of Tumour Developed Nude Rats

30 days after the application of DMSO 1st and 5th compound to tumour developed nude rats, blood was taken intracardiac blood was centrifuged to obtain serum and biochemical parameters were studied.

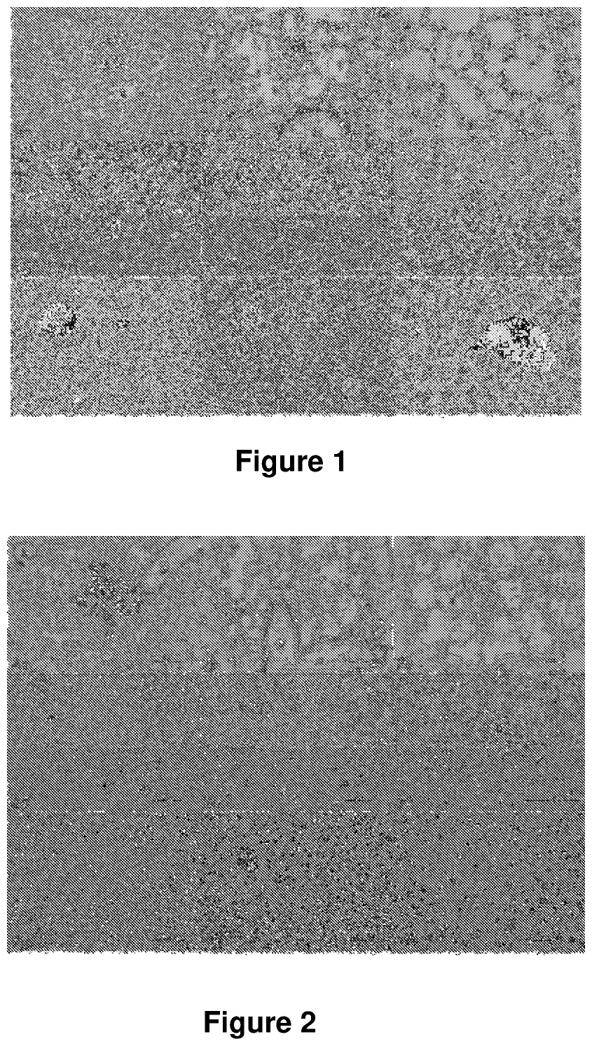

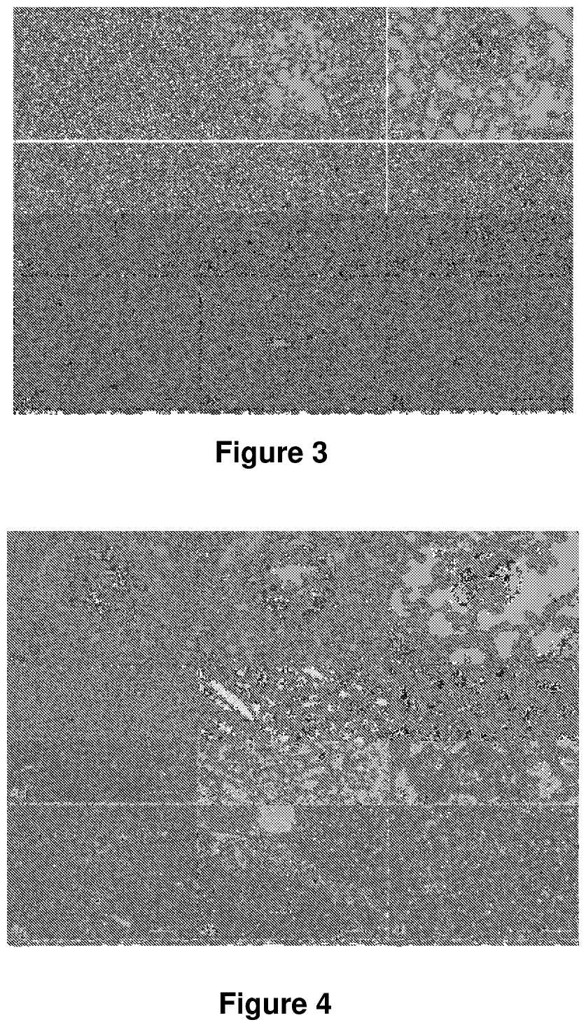

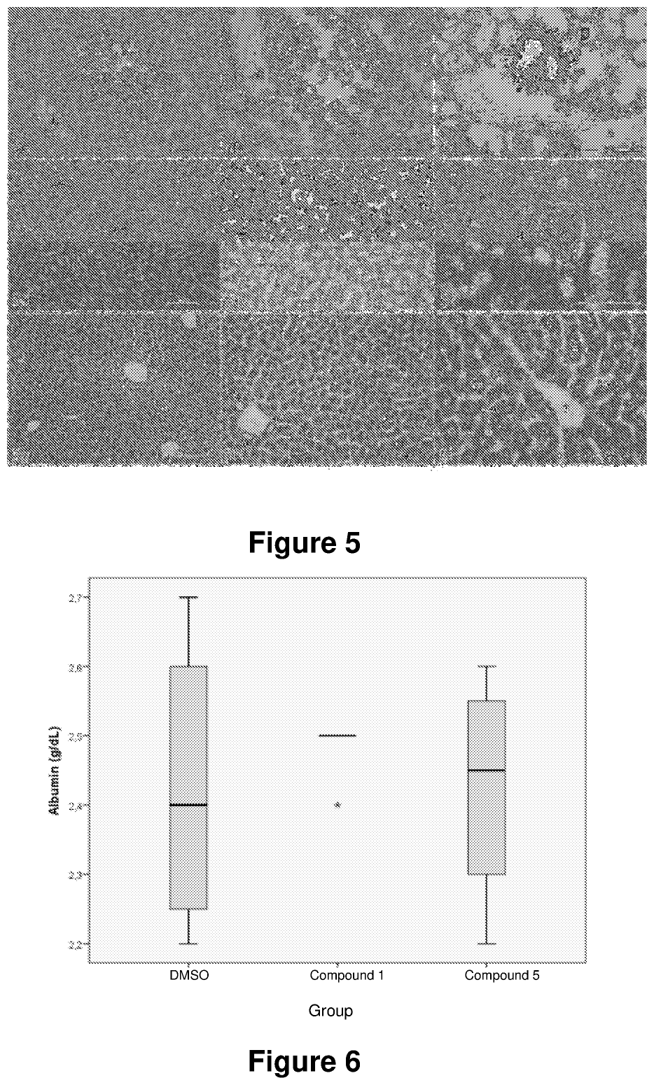

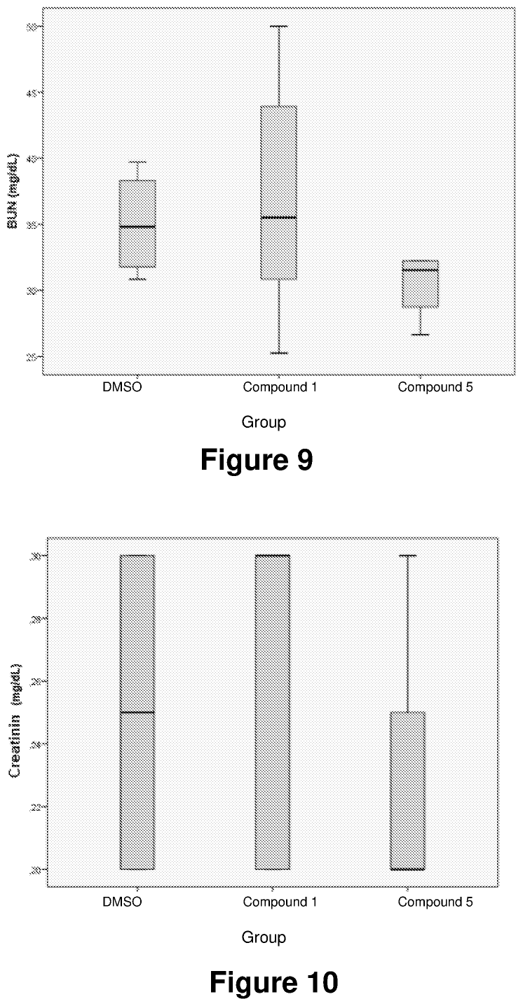

Table 4 shows the results of parameters giving general information about liver and kidney functions based on biochemicals from serums obtained from nude rats regarding DMSO 1^{st} and 5^{th} compound. In short, when compared to group where DMSO is used as solvent, chemicals have caused no damage in parameters showing kidney functions (BUN and Cr) and parameters displaying liver cellular functions and synthesis activities (AST, ALT and albumin). (FIGS. 6-10)

In the light of the results, display of no toxic effect by synthesized compounds on liver and kidney functions, the most important two organs of biotransformation and elimination steps and no difference arising from solvent give information about safety and effectiveness of the compounds.

TABLE 4
Albumin, ALT, AST BUN and Creatinine levels of nude rats administered DMSO
1^{st} and 5^{th} compounds (There is not difference between groups (p > 0.05))

| | | 1^{st} compound | 5. Compound | |
|---|---|---|---|---|
| | DMSO (n = 4) | (n = 5) | (n = 4) | p |
| ALBUMIN | 2.4(2.2-2.7) | 2.5(2.4-2.5) | 2.45(2.2-2.6) | 0.863 |
| ALT | 35(28-68) | 28(22-31) | 36(24-49) | 0.208 |
| AST | 126.5(69-265) | 107(70-157) | 127(82-190) | 0.833 |
| BUN | 34.82(30.84-39.72) | 35.51(25.23-50) | 31.54(26.64-32.24) | 0.371 |
| CREATININ | 0.25(0.2-0.3) | 0.3(0.2-0.3) | 0.2(0.2-0.3) | 0.594 |

The Effect of Benzimidazole Metal Complex Compounds Containing Zinc (1^{st} and 5^{th} Compound) on Expression Levels of Some Proteins in Tissues of Tumour Developed Nude Rats

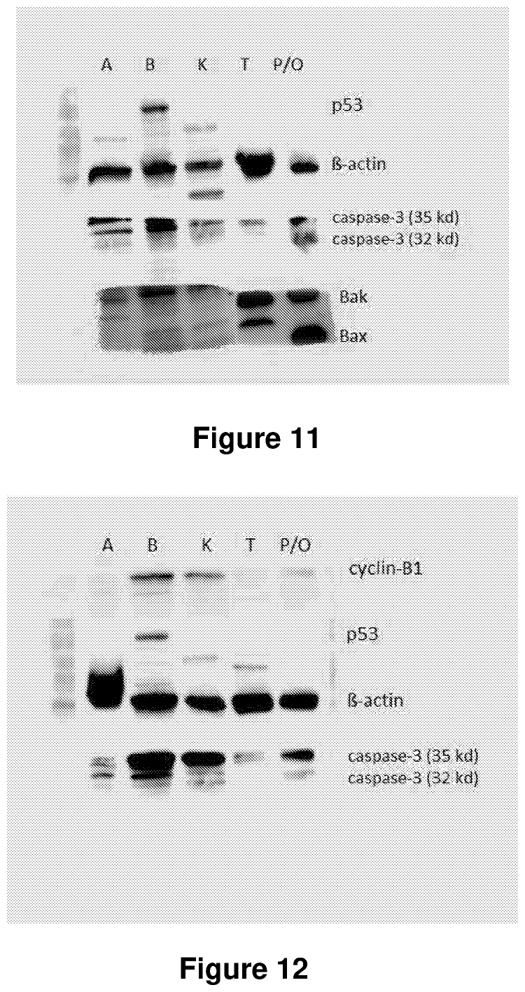

It is seen that p53 protein is expressed in kidney tissue of nude rat injected with DMSO, solvent of compounds. It is seen that caspase 3 is expressed in all examined tissues, that proapoptotic proteins Bak, Bax are expressed in tumour and peritoneum/omentum tissues. (FIGS. 11-13)

It is seen that Cycline B1 synthesized late in S phase of Mitosis and degraded in anaphase is expressed in kidney and liver tissue of 1^{st} compound administered nude rat and p53 is expressed in kidney tissue. Although lower in lung and tumour tissues, caspase 3 is expressed in all tissues. (FIG. 12)

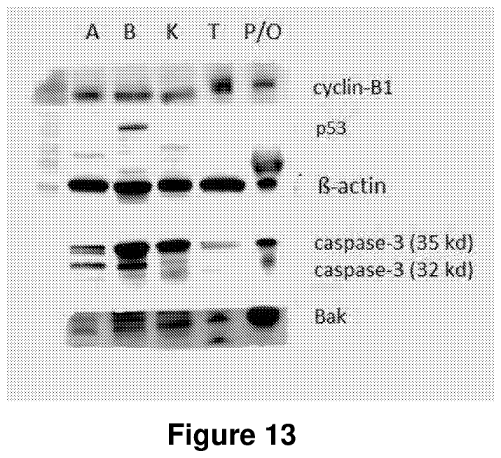

It is seen that cycline B1 is expressed in all examined tissues of nude rat administered compound 5, that only p53 is expressed in kidney tissue. Although lower in tumour tissue, caspase 3 protein is expressed in all of examined tissues. On the other hand, Bak protein which is proapoptotic protein is more in P/O tissue whereas is expressed in all examined tissues. (FIG. 13)

As a result of this invention, it is discovered that tumour size is reduced as a result of administering benzimidazole metal complex compounds containing zinc. Also different from the group where cisplatin is applied, histopathological damage findings were not observed in lung, liver and kidney tissues. It is discovered that when compared to group where DMSO is used as solvent, 1^{st} and 5^{th} compounds have not caused damage in parameters showing kidney functions (BUN and Cr) and parameters displaying liver cellular functions and synthesis activities (AST, ALT and albumin). Display of no toxic effect by synthesized compounds on liver and kidney functions, the most important two organs of biotransformation and elimination steps and no difference arising from solvent give information about safety and effectiveness of the compounds.

1. Use of antitumoural effective benzimidazole metal complexes comprising zinc in treatment of cancer.

2. A compound according to claim 1, characterized in that; it is selecting from 1-(4-chlorobenzyl)benzimidazole and 1-(4-metilbenzyl)benzimidazole zinc complexes.

3. The compound according to claim 1, characterized in that; used in in vivo cancer treatment.

4. A pharmaceutical composition comprising of zinc based benzimidazole metal complex compounds characterized in that; used in cancer treatment.
